(19) United States
(12) Patent Application Publication
(10) Pub. No.: US 2023/0405176 A1
(43) Pub. Date: Dec. 21, 2023

(54) METHOD FOR PRODUCING WOUND DRESSINGS ON THE BASIS OF PHOSPHOLIPID-CONTAINING NANODISPERSIONS

(71) Applicant: PROF4SKIN GMBH, Greven (DE)

(72) Inventor: Rolf DANIELS, Rottenburg (DE)

(21) Appl. No.: 18/034,802

(22) PCT Filed: Nov. 1, 2021
(86) PCT No.: PCT/DE2021/000177

§ 371 Date: May 1, 2023

(30) Foreign Application Priority Data
Oct. 30, 2020 (DE) 10 2020 006 675.5

(51) Int. Cl. A61L 15/20; A61L 15/40; A61L 15/24; A61L 15/34; A61L 15/42; A61L 15/44

(52) U.S. Cl. CPC A61L 15/20; A61L 15/40; A61L 15/24; A61L 15/34; A61L 15/425; A61L 15/44; A61L 2400/12

(57) ABSTRACT

The present invention relates to a method for producing a wound dressing on the basis of phospholipid-containing nanodispersions.

### BACKGROUND OF THE INVENTION

The present invention relates to a method for producing a wound dressing on the basis of phospholipid-containing nanodispersions. It is known to cover wounds in the skin of humans or animals with wound dressings. These are intended to absorb blood and wound exudate and prevent the penetration of foreign bodies. Depending on the design, wound dressings can ensure a moist wound climate or reduce pain, promote wound healing, or have an antimicrobial effect due to the substances they contain. A simple and widely used wound dressing consists of a cotton fabric and adhesive tape and is known as a sticking plaster. Newer wound dressings contain, for example, alginates, hydrogels, or hydrocolloids. Such wound dressings usually have to be fixed to the uninjured skin, for example by films that are themselves coated with acrylate adhesives.

Despite all the developments in the field of wound dressings, there is still a need for wound dressings with improved properties.

WO 2019/243988 A1 describes nanodispersions of phospolipids formed into fiber shapes using electrospinning, which can be used for producing wound dressings.

The present invention describes alternative manufacturing processes for producing wound dressings from the nanodispersions shown in principle in WO 2019/243988 A1.

Further state of the art is found in the following documents:
Choudhury et al., Recent Update on Nanoemulgel as Topical Drug Delivery System, Journal of Pharmaceutical Sciences, Vol. 06, 7, Jul. 2017, p. 1736-1751 describes trans-dermal drug delivery systems based on nanoemulgels.
W02016/198238 A1 describes a material containing a hydrogel and nanoparticles with adjustable porosity for the formation of cell cultures.
Hajialyani et al., Natural product-based nanomedicines for wound healing purposes, Internat. Journal of Nanomedicine 2018, 13, p. 5023-5043 describes various hydrogel-based nanoparticulate systems for wound healing.

### SUMMARY OF THE INVENTION

The present invention relates to a method for producing wound dressings from a phospholipid-containing nanodispersion comprising the following process steps:
a) mixing at least one phospholipid with at least one pharmaceutically acceptable oil and water,
b) performing an emulsification by rotor-stator or ultrasonic emulsification or by high-pressure homogenization of the mixture to obtain a dispersion, wherein at least 90% of the particle droplets have a diameter of less than 10 μm (preferably <1 μm),
c) mixing the dispersion from step b) with the aqueous solution of a pharmaceutically acceptable polymer,
d) layered application of the resulting polymer solution with dispersed particles on a carrier,
e) drying at an elevated temperature, reduced pressure, and/or freeze-drying,
f) optionally mechanical separation of the layer and/or separation from the carrier, according to claim 1.

Further advantageous embodiments of the invention are subject matter of the subclaims.

### DETAILED DESCRIPTION OF THE INVENTION

According to process step a), at least one phospholipid is first mixed with at least one pharmaceutically acceptable oil and water. In principle, all known phospholipids can be used as phospholipids. Preferred phospholipids are phosphatidylcholine, phosphatidylethanolamine, phosphatidylinositol, phosphatidyl-serine, or mixtures thereof. Particularly preferred phospholipids are hydrogenated phospholipones, such as those available under the name “Phospholipon® 90H” or “Phospholipon 80H”. These include mainly hydrogenated phosphatidylcholine. Other commercially available products made from phospholipids for pharmaceutical purposes, especially from hydrogenated phospholipids, can also be used successfully.

The pharmaceutically acceptable oil can generally be selected from the wide variety of known vegetable oils. Castor oil, corn oil, coconut oil, flaxseed oil, olive oil, peanut oil, or mixtures thereof have proven particularly useful.

The mixture from process step a) usually contains:
approx. 1-15% phospholipid, preferably 2.5-9%,
approx. 1-10% pharmaceutically acceptable oil, preferably 1-5% water ad 100%.

Optionally, the aqueous mixture may also contain 0.05 to 5 wt. % birch extract. For this purpose, such birch extract is particularly preferred, which contains the following components (data in percent by weight (wt. %)):

| | | |
|---|---|---|
| Betulin | 74-85 | wt. %, |
| Lupeol | 1.0-4.0 | wt. %, |
| Betulinic acid | 3.0-5.0 | wt. %, |
| Erythrodiol | 0.3-2.8 | wt. % |

and optionally other birch extract components, in particular oleanolic acid, betulinic acid, betulinic acid methyl ester 2-13 wt. %.

One advantage of the birch extract-containing nanodispersion is that the pharmaceutically active components of the birch bark extract are dispersed into very fine particles or droplets, resulting in an improved bioavailability. Such nanodispersion can be applied directly to wounds or infected skin, for example by spraying it onto the affected body part. The nanodispersion according to the present invention is superior to an oleogel in terms of handling properties. It is a liquid that can be sprayed or spread even on infected skin areas.

The aqueous mixture of phospholipid and pharmaceutically acceptable oil and optional birch extract described above is mixed using a suitable mixer-homogenizer to produce a pre-dispersion. This pre-dispersion is then further treated to reduce the desired particle size of the individual droplet particles from an average droplet particle size of <10 μm, if necessary, to a submicron size of about <1 μm and preferably below 400 nm. This can be done by intensive shearing using a rotor-stator homogenizer or an ultrasonic emulsification process, which are very efficient in reducing droplet size.

Alternatively, high-pressure homogenization can be used, e.g., a high-pressure homogenizer of the piston gap type, to produce nanoemulsions with extremely low particle sizes down to a few nanometers. Ultrasonic application and microfluidization are other well-known and sufficiently described methods for producing nanoemulsions.

It is well known that such dispersions are subject to a size distribution of the particles. Depending on the circumstances of the manufacturing process, the size distribution of the particles (especially the median value and the spread) may vary. For the present method, it has been found that the median value of the particle sizes should be in the nanometer range, i.e., smaller than 1,000 nm, preferably smaller than 800 nm, particularly preferably smaller than 800 nm. In individual cases, however, a dispersion with a median value of the size distribution of 5,000 nm (5 μm) may be sufficiently stable to achieve the desired result. In any case, at least 90% of the particles should have a size smaller than 10,000 nm (10 pm), preferably 90% of the particles should have a size smaller than 5,000 nm (5 μm), particularly preferably 90% of the particles should have a size smaller than 1,000 nm (1 μm). All dispersions defined above are referred to as nanodispersions in this document. The above-mentioned methods for producing dispersions with the above-mentioned properties, in particular for achieving the desired particle size distributions, are known in principle and therefore do not require detailed explanation here. Reference is made, for example, to the monograph “Emulgiertechnik”, B. Behr's Verlag, 3rd ed. 2012, ISBN 978-3-89947-869-3, in particular, Chapter VII. 5.

The pre-emulsion is converted into a nanodispersion in step b) of the process. The particle droplets contained in this nanodispersion have a size distribution in the lower micrometer range. At least 90% of the particle droplets must have a diameter of smaller than 10 micrometers.

For the conversion of the pre-emulsion generated in step a) into a nanodispersion, various methods are available from the prior art, for example, the use of a rotor-stator homogenizer with high shear (e.g., gear disperser or colloid mill), by application of ultrasound, by high-pressure homogenization, or by microfluidization. All these methods are sufficiently described in the prior art so that no further explanations are necessary here.

According to the invention, the nanodispersion formed is then mixed with an aqueous polymer solution. In this way, extremely small droplets of the nanodispersion are dispersed within the carrier polymer. The dispersant particles of the birch bark extract are finely dispersed in the polymer matrix, and an ultra-homogenization step may also be helpful.

The polymers usable for the preferred embodiment must be pharmaceutically acceptable. Such polymers may be selected from polyethylene oxide, polyvinyl alcohol, polyvinyl acetate, polyvinyl pyrrolidone, hyaluronic acid, alginates, carrageenan, xanthan gum, cellulose derivatives such as carboxymethyl cellulose, sodium methyl cellulose, ethyl cellulose, hydroxyethyl cellulose, hydroxypropyl cellulose, hydroxypropyl methyl cellulose, hydroxypropyl methyl cellulo-sephthalate, cellulose acetate phthalate, starch derivatives such as hydroxyethyl starch, sodium starch glycolate, chitosan and derivatives, albumin, gelatin, collagen, polyacrylates, and derivatives thereof. Particularly preferred are those polymers that are readily soluble on a wound without undesirable properties. Polyvinyl alcohol or poly(lactide-co-glycolide) are particularly preferred. The most preferred is the hydrophilic polymer polyvinyl alcohol (PVA), which has already been approved by the FDA for human use. PVA polymers have unique properties such as good chemical resistance, thermal stability, biodegradability, biocompatibility, and non-toxicity, which make them suitable for wound dressings.

As a rule, an aqueous solution of the polymer is prepared. The solution contains about 5-20% by weight of the polymer, preferably about 10%. The aqueous polymer solution is mixed with the nanodispersion from process step b) in a weight ratio of 25:75 to 75:25. A preferred mixing ratio is 60-70% of the polymer solution to 40-30% of the nanodispersion.

In step d) of the process, the polymer-dissolved nanodispersion produced in this way is applied in layer form to a carrier. Suitable supports are, for example, aluminum foil, plastic films, glass, cotton fabrics, or fleeces, or polymer fabrics or fleeces. Optionally, the layer is mechanically smoothed and at the same time excess dispersion is removed, e.g., by using a squeegee.

The nanodispersion can also be applied to the carrier in such a way that the nanodispersion is first foamed by introducing gases (e.g., with the introduction of propane/butane; N20 or dimethyl ether under pressure) and the foam is then applied uniformly to the carrier.

In step e) of the process, drying of the nanodispersion layer takes place. This drying can be carried out at an elevated temperature and/or at a reduced pressure. The temperature should not exceed 60 degrees Celsius. Typical pressure ranges for drying are 10 to 100 mbar, although lower pressures are certainly possible.

Alternatively, drying can also be carried out by freeze-drying.

The dried nanodispersion should have a layer thickness of 0.05 to 5 mm.

After drying, the generated layer is typically mechanically cut or punched into pieces. The size and shape of the individual pieces can vary. Typical sizes are 1 to 100 square centimeters in size and of various shapes (e.g., round, oval, or rectangular). However, larger pieces can also be created as needed. These can optionally be adapted to the geometry of the wounds.

In this case, cutting into pieces can be done with or without a carrier. For example, the layer can be detached from the carrier and then cut. Alternatively, the layer can be cut with a carrier film, and the film as a carrier can then be released immediately before use.

As part of the process, sterilization may of course still be carried out, for example by heating, sterile filtration, and/or irradiation of the aqueous mixture prepared in step 1. Alternatively or additionally, the wound dressing produced at the end of the process can also be sterilized thermally or by irradiation.

The dried nanodispersion produced in accordance with the process is suitable as a wound dressing. Since the layer itself has only low adhesive properties, it is usually fixed to the wound or wound edges by means of another film with adhesive properties (adhesive layer) or a suitable adhesive.

### EXAMPLES

The following examples show further details of the process according to the invention without being restrictive. The person skilled in the art can, based on the information shown here, carry out further developments without having to be inventive.

### Example 1: Base Mixtures for Producing Nanodispersions

| | |
|---|---|
| 1a) Phospholipon 90 H | 2.5% |
| Sunflower oil | 1.0% |
| Birch extract | 0.5% |
| Water | 96.0% |
| 1b) Phospholipon 90 H | 2.5% |
| Sunflower oil | 1.0% |
| Water | 96.5% |
| 1c) Phospholipon 90 H | 8.0% |
| Sunflower oil | 10.0% |
| Birch extract | 5.0% |
| Water | 77.0% |
| 1d) Phospholipon 90 H | 8.0% |
| Sunflower oil | 10.0% |
| Water | 82.0% |
| 1e) Phospholipon 80 H | 2.5% |
| Sunflower oil | 1.0% |
| Birch extract | 0.5% |
| Water | 96.0% |
| 1f) Phospholipon 80 H | 2.5% |
| Sunflower oil | 1.0% |
| Water | 96.5% |
| 1g) Phospholipon 80 H | 8.0% |
| Sunflower oil | 10.0% |
| Birch extract | 5.0% |
| Water | 77.0% |
| 1h) Phospholipon 80 H | 8.0% |
| Sunflower oil | 10.0% |
| Water | 82.0% |
| 1i) Phospholipon 90 H | 2.5% |
| Corn oil | 1.0% |
| Birch extract | 0.5% |
| Water | 96.0% |
| 1j) Phospholipon 90 H | 2.5% |
| Coconut oil | 1.0% |
| Water | 96.5% |
| 1k) Phospholipon 80 H | 8.0% |
| Olive oil | 10.0% |
| Birch extract | 5.0% |
| Water | 77.0% |
| 1l) Phospholipon 80 H | 8.0% |
| Linseed oil | 10.0% |
| Water | 82.0% |

### Example 2

The mixture prepared according to Example 1 is first converted into a nanodispersion as follows:

The mixture of phospholipid and pharmaceutically acceptable oil and optional birch extract from process step a) is mixed in a mixer-homogenizer and in this way, a pre-dispersion is prepared. This pre-emulsion is then further treated (at least 3, preferably 8 cycles at 100 MPa, 70° C.) using high-pressure homogenization (Emulsiflex C-3, Avestin, Mannheim, Germany) to reduce the particle size of the individual droplet particles from an average droplet particle size of >10 μm to a submicron size of about <1 μm and preferably below 400 nm.

The nanodispersion is then intensively mixed with a polymer solution (10% PVA solution) (e.g. mixer-homogenizer of the Bekomix or Frymacoruma types) and further processed as follows:

### Variant 1)

The polymer-dissolved nanodispersion is applied (preferably sprayed) to a carrier layer, e.g., a textile fabric or fleece, and then dried at an elevated temperature, e.g., 60° C., with or without the application of a vacuum.

The result is, for example, an adhesive bandage that is provided with an active layer.

### Variant 2)

The polymer-dissolved nanodispersion is applied (poured or sprayed) to a suitable substrate, e.g., carrier film, and then dried at an elevated temperature, e.g., 60° C., with or without the application of a vacuum.

The resulting film (film thickness approx. 0.1-0.2 mm) is separated from the backing, cut to a suitable size if necessary, and can now be used as a wound dressing on its own in isolation or in combination with other materials used for wound dressings and/or an adherent layer to form composite wound dressings.

### Variant 3)

The polymer-dissolved nanodispersion is converted into foam by introducing air or another suitable gas through nozzles or by mechanical incorporation of air or another suitable gas. This foam is applied (spread, poured, or sprayed) to a suitable substrate, e.g., carrier film, and then dried at an elevated temperature, e.g., 60° C., with or without applying a vacuum.

The resulting porous film is separated from the substrate, cut to a suitable size if necessary, and can now be used as a wound dressing on its own in isolation or in combination with other materials used for wound dressings and/or an adherent layer to form composite wound dressings.

### Variant 4)

The polymer-dissolved nanodispersion is subjected in a suitable layer thickness to a conventional freeze-drying process at a temperature of <−20° C. and then freeze-dried.

The resulting porous, sponge-like layer is separated from the substrate, e.g., carrier film, cut to a suitable size if necessary, and can then be used as a wound dressing on its own in isolation or combined with other materials used for wound dressings and/or an adherent layer to form composite wound dressings.

### Example 2a

As an alternative to Example 2, a nanodispersion can be prepared as follows:

Phospatidylcholine and birch bark extract are dispersed in water at 70° C. and stirred under vacuum for 30 min. This is followed by homogenization for 10 min (rotor-stator >10 m/s). With further stirring and under vacuum, sunflower oil is added and homogenized for another 30 min (rotor-stator >10 m/s). The phase is cooled to 40° C. The polymer solution (10% PVA solution) is added and intensively mixed (e.g. mixer-homogenizer of the types Bekomix or Frymacoruma). The polymer-dissolved nanodispersion can be further processed as described in Example 2 (variants 1-4).

### Example 3

To demonstrate the activity of wound dressings according to the invention, an ex vivo wound healing test for pigs can be performed. For this purpose, pig ears from a slaughterhouse (for human consumption) are delivered to the laboratory directly after slaughter, cleaned, and disinfected. Then, 6 mm punch biopsies are taken from the earlobes and fat and subcutaneous tissue are removed. Consequently, wounds were formed by removing the epidermis and upper dermis in a central area of 7.1 mm². Then, the ex vivo wound healing model thus formed is placed in culture dishes, dermis at bottom, on gauze and incubated with Dulbecco's modified Eagle's medium supplemented with hydrocortisone, 2% fetal calf serum, penicillin, and streptomycin at the air-liquid interface. 4 cm² of wound dressing was applied immediately after wounding, and models were incubated for 48 h at 37° C. and 5% CO₂. Further steps included snap freezing, cryostat sections of the central parts of the wound healing models are identified with a ruler in the microscope, and—by checking the total length of the wound during evaluation—stained with hematoxylin and eosin. The wound healing process (re-epithelialization) is assessed by measuring the distance between the wound margin and the tip of the regenerated epidermis with a microscope.

In the example, the wound was not treated as a control at all. An oleogel was used as a comparison to the state of the art. A slight improvement was observed. Furthermore, a polyvinyl alcohol mat without birch bark extract (PVA mat) was used. It can be seen that the healing properties are better than with the control and even better than with oleogel.

### BRIEF DESCRIPTION OF THE DRAWING

FIG. 1 shows the results of the tests performed: Column 1 (far left) shows the untreated control; Column 2 shows the treatment with oleogel (corresponding to the approved finished drug Episalvan); Column 3 shows treatment with an electrospun wound dressing containing birch extract according to WO 2019/243988 A1; Column 4 shows treatment with an electrospun placebo wound dressing; and Column 5 shows the treatment with the TE-containing film from FIG. 1.

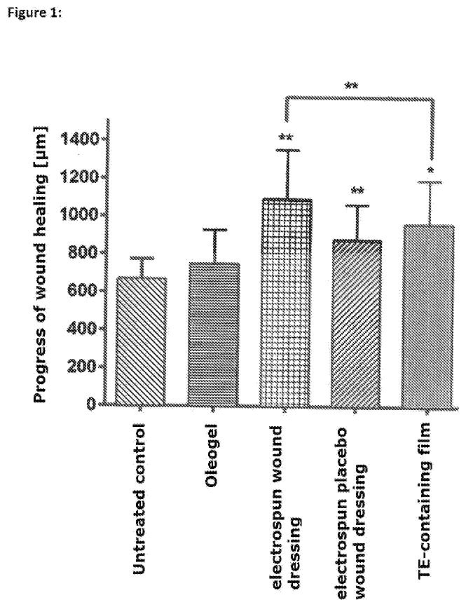

1. A method for producing wound dressings on the basis of phospholipid-containing nanodispersions, comprising the following method steps:
a) mixing at least one phospholipid with at least one pharmaceutically acceptable oil and water,
b) performing an emulsification by rotor-stator or ultrasonic emulsification or by high-pressure homogenization of the mixture to obtain a dispersion, wherein at least 90% of the particle droplets have a diameter of less than 10 μm,
c) mixing the dispersion with the aqueous solution of a pharmaceutically acceptable polymer,
d) layered application of the resulting polymer solution with dispersed nanoparticles on a carrier,
e) drying at an elevated temperature, reduced pressure, and/or freeze-drying,
f) optionally mechanical separation of the layer and/or separation from the carrier.

2. The method of claim 1, wherein the phospholipid is selected from phosphatidytcholine, phosphatidylethanolamine, phosphatidytinositol, phosphatidyl-serine, hydrogenated phospholipids (especially hydrogenated phosphatidylcholine} or mixtures thereof.

3. The method of claim 1, wherein at least one pharmaceutically acceptable oil is selected from sunflower oil, castor oil, corn oil, coconut oil, flaxseed oil, olive oil, peanut oil, or mixtures thereof.

4. The method of claim 1, wherein the mixture of phospholipid with oil and water contains 0.05 to 5% by weight of birch extract.

5. The method of claim 4, wherein the birch extract comprises the following components, calculated as weight percent:

| | | |
|---|---|---|
| Betulin | 74-85 | wt. %, |
| Lupeol | 1.0-4.0 | wt. %, |
| Betulinic acid | 3.0-5.0 | wt. %, |
| Erythrodiol | 0.3-2.8 | wt. % |

and optionally other birch extract components, in particular oleanolic acid, betulinic acid, betulinic acid methyl ester 2-13 wt. %.

6. The method of claim 1, wherein the nanodispersion is produced
by application of a mixing apparatus with high shear,
by application of ultrasound,
by high-pressure homogenization, or
by microfluidization.

7. The method of claim 1, wherein the pharmaceutically acceptable polymer is selected from polyethylene oxide, polyvinyl alcohol, polyvinyl acetate, polyvinyl pyrrolidone, hyaluronic acid, alginates, carrageenan, xanthan gum, cellulose derivatives, sodium methyl cellulose, ethyl cellulose, hydroxyethyl cellulose, hydroxypropyl cellulose, hydroxypropyl methyl cellulose, hydroxypropyl methyl cellulose phthalate, cellulose acetate phthalate, starch derivatives, sodium starch glycolate, chitosan, and derivatives, albumin, gelatin, collagen, polyacrylates, and derivatives thereof, polyvinyl alcohol or poly(lactide-co-glycolide).

8. The method of claim 1, wherein the nanodispersion dispersed in the polymer is applied to a carrier, the carrier being selected from aluminum foil, plastic film, glass, cotton fabrics or fleeces, or polymer fabrics or fleeces.

9. The method of claim 1, wherein the nanodispersion dispersed in the polymer is applied to a carrier with foaming, the carrier being selected from aluminum foil, plastic foil, glass, cotton fabrics or fleeces, or polymer fabrics or fleeces.

10. The method of claim 1, wherein characterized by that drying of the nanodispersion dispersed in the polymer is carried out at an elevated temperature and/or at a reduced pressure.

11. The method of claim 1, wherein drying of the nanodispersion dispersed in the polymer is carried out by freeze-drying.

12. The method of claim 1, wherein the dried layer of nanodispersion dispersed in the polymer has a layer thickness of 0.1 to 5 mm.

13. The method of claim 1, wherein after drying, mechanical comminuting of the dried layer into pieces of 1-100 cm² is carried out.

14. The method of claim 1, wherein after drying, a separation of the carrier is carried out.

15. A wound dressing, produced according to the method of claim 1.

16. Use of the dried nanodispersion dispersed in the polymer, produced according to the method of claim 1.
